(19) United States
(12) Patent Application Publication
(10) Pub. No.: US 2018/0310513 A1
(43) Pub. Date: Nov. 1, 2018

(54) DELOSPERMA PLANT AND A METHOD FOR PRODUCING THE DELOSPERMA PLANT

(71) Applicant: Koichiro NISHIKAWA, Kanagawa (JP)

(72) Inventor: Koichiro NISHIKAWA, Kanagawa (JP)

(21) Appl. No.: 15/738,307

(22) PCT Filed: Jul. 18, 2017
(86) PCT No.: PCT/JP2017/026588

§ 371 Date: Dec. 20, 2017

(30) Foreign Application Priority Data
Mar. 6, 2017 (JP) PCT/JP2017/009855

(51) Int. Cl. A01H 6/00; A01H 5/02

(52) U.S. Cl. CPC A01H 6/00; A01H 5/02

(57) ABSTRACT

The Delosperma plant (1a) is characterized by being capable of maintaining a flowering state in case where an air temperature is more than a prescribed temperature, even when it gets dark at nightime, wherein the prescribed temperature is 15 degree Celsius. This configuration shows that it is possible to produce the Delosperma plant (1a) with a characteristic of having longer blooming hours than conventional varieties of Delosperma.

### BACKGROUND OF THE INVENTION

### 1. Field of the Invention

The present invention relates to a Delosperma plant which has a unique flowering physiology and a method for producing the Delosperma plant.

### 2. Description of the Related Art

Delosperma belongs to the family Aizoaceae, and is native to South Africa in general. The name of the genus is derived from the Greek words delos (visible) and sperma (seed). Delosperma is an herbaceous perennial, and has succulent thick leaves. Its stem extends horizontally. Delosperma is widely used for open cultivation, container and landscape purposes in Japan owing to its cold and drought tolerance.

Delosperma usually has small flowers, which are similar to daisy flowers, while some Delosperma species have brightly-colored flowers and some other species bloom in spring and last until the beginning of winter. For example, Delosperma congestum, which is a kind of Delosperma species, has vivid yellow flowers and thick deep green leaves. This Delosperma congestum grows in colonies and gradually spreads out on the ground. And Delosperma cooperi (also known as its generic names HANARANZAN and REIKOU), which is an herbaceous perennial, has pink or crimson flowers and dense branches, and spreads quickly and its roots survive during winter.

In addition, there is a genus of plants similar to Delosperma, known as Lampranthus; however, this genus can be easily distinguished from Delosperma, because Lampranthus plants bloom only for a short period in spring, and the plants are sensitive and intolerant to cold.

The inventor of the present application has already bred many new cultivars of Delosperma plants and owns a plurality of plant patents in the U.S. For example, the varietal denomination of the new cultivar bred by the inventor of the present application is ‘Jewel of Desert Rosequartz’ (refer to U.S. Plant Pat. No. 23,452); ‘Jewel of Desert Ruby’ (refer to U.S. Plant Pat. No. 23,453); ‘Jewel of Desert Garnet’ (refer to U.S. Plant Pat. No. 23,471); ‘Jewel of Desert Moon Stone’ (refer to U.S. Plant Pat. No. 23,491); ‘Jewel of Desert Topaz’ (refer to U.S. Plant Pat. No. 23,492); ‘Jewel of Desert Peridot’ (refer to U.S. Plant Pat. No. 23,566); ‘WOW (Wheels of Wonder) DRW5’ (refer to U.S. Plant Pat. No. 25,572); ‘WOWDRW7’ (refer to U.S. Plant Pat. No. 25,599); ‘WOWDOY3’ (refer to U.S. Plant Pat. No. 25,600); ‘WOWDAY2’ (refer to U.S. Plant Pat. No. 25,645); ‘WOW2011-1’ (refer to U.S. Plant Pat. No. 25,684); ‘WOWDRY1’ (refer to U.S. Plant Pat. No. 26,157); ‘DSAM13-1’ (refer to U.S. Plant Pat. No. 27,013); ‘DSAA13-1’ (refer to U.S. Plant Pat. No. 27,014); and ‘DSAB13-1.’ (refer to U.S. Plant Pat. No. 27,056). Note that the translated term of “Jewel of Desert,” which constitutes one of the names of new cultivars of Delosperma, has been registered as “SABAKU NO HŌSEKI (registered trademark)” in Japan.

Furthermore, the inventor of the present application has been providing these new cultivars of Delosperma to both several foreign and domestic corporations (refer to product catalogs available at Non-patent Reference 1 “Dümmen Orange Perennials 2017.”

Retrieved Dec. 8, 2016, from https://issuu.com/dummenorange/docs/2017_catalog_Ir_perennials, Non-patent Reference 2; Delosperma “Jewel of Desert.” Retrieved Dec. 8, 2016, from http://www.delosperma.com/jewelofdesert/japanese.html, and Non-patent Reference 3; “Proven winners Wheels of Wonder.” Retrieved Dec. 8, 2016, from https://www.soonerplantfarm.com/plant/ice-plant-wheels-of-wonder-fire-wonder/)

### BRIEF SUMMARY OF THE INVENTION

Next, the flowering physiology of conventional Delosperma will be explained. Since Delosperma is a desert plant and strong against dryness, Delosperma starts to bloom by getting strong light during daytime, and starts to close its flowers during nightime in order to protect the flowers from strict desert climate such as severe cold, condensation, and so on. In other words, normally speaking, Delosperma starts to close its flowers by the time it gets a little dark in the evening, and keeps the flowers closing during the night, and does not open the flowers by the time it gets bright in the morning. For example, although it depends on the season and the region it is planted on, the flowering time is slow, and the flower opens up at 9 to 10 AM o'clock in the morning, and it is often seen that the flower closes around 3 o'clock in the evening. Also, rain and cloudy days often have flowers closed all day. In addition to that, the flowers often close the whole day during rainy and cloudy weather conditions

Due to the described flowering physiology of Delosperma plants, it results to the Delosperma plants being in their closed state, whenever they are being sold in flower shops. Specifically, the light in flower shops is not enough bright or the temperature in flower shops is not enough warm, so that the conventional Delosperma keeps the flowers closing during open hours of the flower shops. Further, even if it starts to bloom, the flowers start to close around 3 PM. In addition, there is even a case of Delosperma not flowering when placed inside a room or under the shade of a building. This is due to lack of sufficient light and temperature.

Accordingly, despite the attractive colorful appearance of the Delosperma, which is shown in FIG. 20, the flowers keep closing during open hours in flower shops. Therefore, the consumers cannot fully appreciate their appearance, because as it was stated before, the flowers close themselves during the times they are being sold in the stores. As a result of this, the retail sale of Delosperma is not promoted and more sales cannot be expected. Accordingly, it is obvious that the development of new cultivar of Delosperma, which can keep the flowers opening, regardless of the temperature and weather conditions such as a solar radiation value, enhances its commercial value rapidly.

The present invention has been made in view of the abovementioned problems, and has an object to provide a Delosperma plant having longer blooming hours, more specifically, being capable of maintaining a flowering state when it gets dark at nightime, and a method for producing the Delosperma plant.

### DISCLOSURE OF INVENTION

In order to solve the aforementioned issues, the present invention is a Delosperma plant characterized by being capable of maintaining a flowering state in case where an air temperature is more than a prescribed temperature, even when it gets dark at nightime, wherein the prescribed temperature is 15 degree Celsius.

In this Delosperma plant, preferably, wherein the prescribed temperature is 12.5 degree Celsius.

In this Delosperma plant, preferably, wherein the prescribed temperature is 10 degree Celsius.

In this Delosperma plant, preferably, wherein the prescribed temperature is 5 degree Celsius.

In this Delosperma plant, preferably, wherein said dark at nightime indicates a space where illuminance is reduced from daylight to less than 1 luce.

In this Delosperma plant, preferably, wherein a ratio of the flowers, which are capable of maintaining the flowering state, is at least more than 20% of all the flowers per stem.

In this Delosperma plant, preferably, wherein a ratio of the flowers, which are capable of maintaining the flowering state, is at least more than 40% of all the flowers per stem.

In this Delosperma plant, preferably, wherein the Delosperma plant comprises no stamen.

In this Delosperma plant, preferably, wherein the Delosperma plant comprises a female sterile flower, which is unable to generate seeds even after a pistil of the Delosperma plant receives self or non-self pollens.

In this Delosperma plant, preferably, wherein the Delosperma plant forms no tuberous root.

In this Delosperma plant, preferably, wherein the Delosperma plant is (a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar, (b) a cultivar produced by crossing species, wherein at least either one of the parents of the species is selected from the Variety A, or (c) a progeny of one of the cultivars (a) and (b).

In this Delosperma plant, preferably, wherein the Delosperma plant is (a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant and then selecting said cultivar, (b) a cultivar produced by crossing species, wherein at least either one of the parents of the species is selected from the Variety B, or (c) a progeny plant of one of the cultivars (a) and (b).

In this Delosperma plant, preferably, wherein the Delosperma plant is one of varieties selected from a Delosperma plant deposited with the international depositary authority under deposit number FERM BP-22335, a progeny of said Delosperma plant, a hybrid plant of the Delosperma plant, and a progeny of the hybrid plant.

In order to solve the aforementioned issues, the present invention is a Delosperma plant obtained by utilizing either one of pollen, ovum, cell, and genetic information relating to DNA and RNA of the abovementioned Delosperma plant.

In order to solve the aforementioned issues, the present invention is a method for producing a Delosperma plant comprising; (a) utilizing either one of pollen, ovum, cell, and genetic information relating to DNA and RNA of the Delosperma plant of claimed above so as to produce the Delosperma plant.

According to the present invention, the Delosperma plant is characterized by being capable of maintaining a flowering state in case where an air temperature is more than a prescribed temperature, even when it gets dark at nightime. For example, the prescribed temperature is 15 degree Celsius. Thus, the Delosperma plant has longer blooming hours than conventional varieties of Delosperma.

### BRIEF DESCRIPTION OF THE DRAWINGS

The present invention will be described hereinafter with reference to the annexed drawing. It is to be noted that the drawing is shown for the purpose of illustrating the technical concepts of the present invention or embodiments thereof, wherein:

FIG. 1 shows Delosperma plants in bloom according to embodiment 1 of the present invention.

FIG. 2 shows Delosperma plants in bloom according to embodiment 1 of the present invention.

FIG. 3 shows Delosperma plants in bloom according to embodiment 1 of the present invention.

FIG. 4 shows Delosperma plants in bloom according to embodiment 1 of the present invention.

FIG. 5 shows Delosperma plants in bloom according to embodiment 1 of the present invention and a conventional cultivar.

FIGS. 6a and 6b show Delosperma plants in bloom according to embodiment 1 of the present invention and a conventional cultivar.

FIG. 7 shows a flowchart to show processes of breeding.

FIG. 8a to FIG. 8f show pictures which explain a biotron.

FIG. 9 shows Delosperma plants in bloom according to Example 3 of embodiment 1.

FIG. 10a to FIG. 10c show Delosperma plants in bloom according to Example 3 of embodiment 1.

FIG. 10d shows a table showing the ratio of Delosperma plants in bloom.

FIG. 11 shows Delosperma plants in bloom according to Example 4 of embodiment 1.

FIG. 12 shows Delosperma plants in bloom according to Example 4.

FIG. 13 shows Delosperma plants in bloom according to Example 4.

FIG. 14 shows Delosperma plants in bloom according to Example 4.

FIG. 15 shows Delosperma plants in bloom according to Example 5 of embodiment 1.

FIG. 16a shows Delosperma sutherlandii.

FIG. 16b and FIG. 16c show the roots of Delosperma sutherlandii.

FIG. 17a shows Delosperma ashtonii.

FIG. 17b and FIG. 17c show the roots of Delosperma ashtonii.

FIG. 18a shows Delosperma plants in bloom according to embodiment 2 of the present invention.

FIG. 18b shows a sectional view of Delosperma plants according to embodiment 2 of the present invention.

FIG. 19 shows Delosperma plants in bloom according to embodiment 2 of the present invention.

FIG. 20a shows conventional Delosperma “15-tai-1” in bloom.

FIG. 20b shows conventional Delosperma “WOW (Wheels of Wonder) DRW5” in bloom.

### DETAILED DESCRIPTION OF THE INVENTION

Embodiments of the present invention, as best mode for carrying out the invention, will be described hereinafter with reference to the drawing. It is to be understood that the embodiments described herein are not intended as limiting, or encompassing the entire scope of, the present invention.

### Embodiment 1

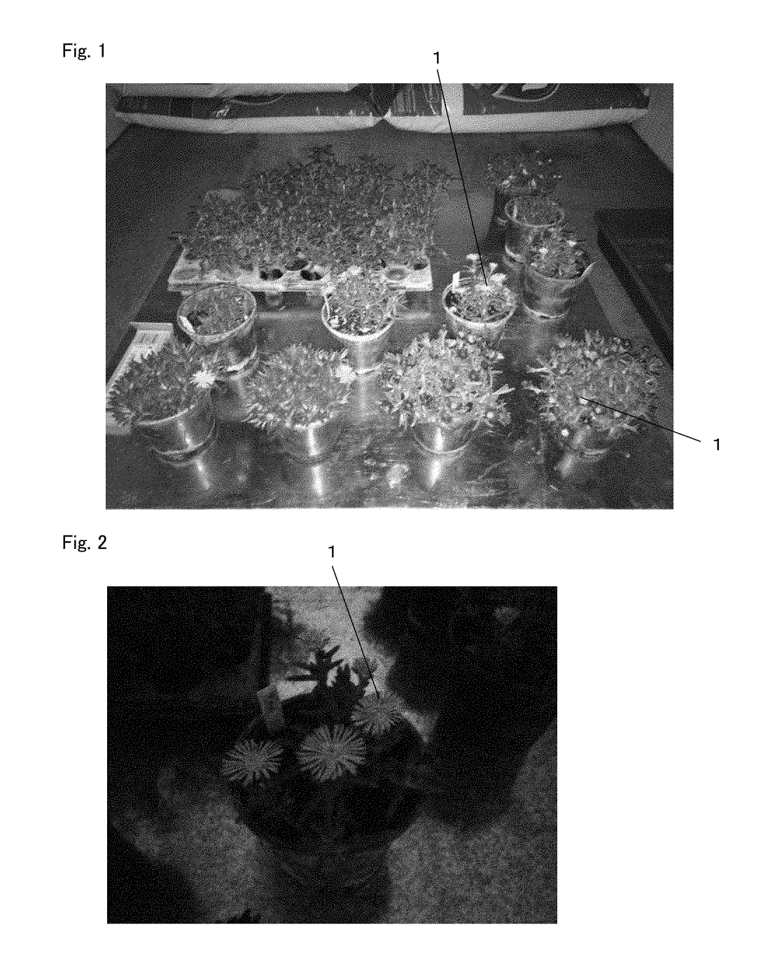

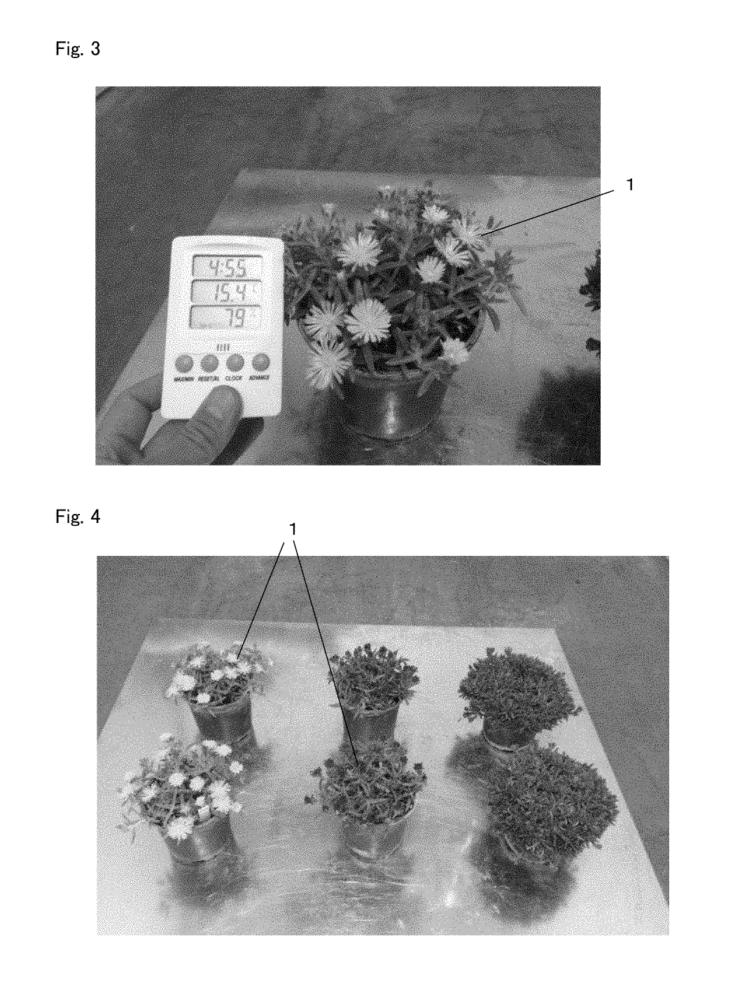

Referring from FIG. 1 to FIG. 17, a Delosperma plant according to Embodiment 1 of the present invention will be described. Generally speaking, Delosperma tends to open its flowers with strong sunlight and high temperature. In other words, the opening/closing of Delosperma flowers is strongly influenced by the strength of light and temperature. The present invention is to provide a new Delosperma plant, which can maintain a flowering state in case where an air temperature becomes comparatively lower (5 to 15° C.), even under conditions when there is no light such as dark at night.

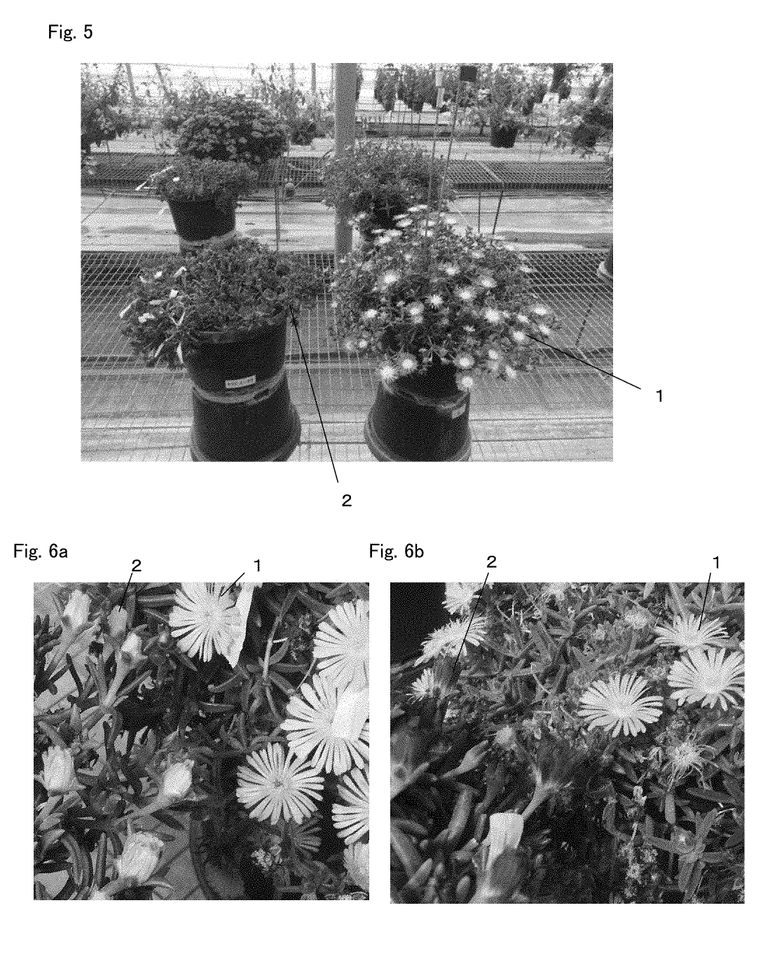

More specifically, the inventor of the present application has successfully obtained the Delosperma plant having longer blooming hours (e.g a characteristic of maintaining a flowering state in case where an air temperature of the room containing the plant is more than a prescribed temperature, even when it gets dark at nightime) through the process of seeding, crossing and selection. As a result of this, the flowers of the Delosperma plant can bloom longer than conventional Delosperma plants. As shown in FIG. 1 to FIG. 4, the Delosperma plant 1 according to Embodiment 1 of the present invention can maintain a flowering state in case where an air temperature is more than a prescribed temperature, even when it gets dark at nightime. FIG. 5 shows the Delosperma plant 1 around 6 PM. This figure shows that the Delosperma plant 1 described herein can maintains its flowering state, while the conventional Delosperma plant 2 has already closed its flowers.

Further, the conventional Delosperma plant 2 shown in FIG. 6a and FIG. 6b has already closed its flowers by the time it gets dark in the evening, or the conventional Delosperma plant 2 has not opened its flowers yet in the morning after breaking dawn. On the contrary, the Delosperma plant 1 shown in FIG. 6a and FIG. 6b according to Embodiment 1 has not closed the flowers even after it gets dark at nightime, or the Delosperma plant 1 has already opened the flowers in the morning when the temperature is more than a prescribed temperature.

Herein, “nightime” means a space where the illuminance is reduced from daylight to less than 1 luce in the description of Embodiment 1, and luce (Ix) is a unit of illuminance and can quantifies light, which is emitted from a light source, and then is reflected from a radiation object such as a plant (Delosperma).

Further, the “flowering (blooming)” of Delosperma flower is a concept that includes more than 50% opening, when 100% opening is defined as from the fully closed to fully opened position of a petal. Herein, the concept is thought to be the flower blooming (opening), because there are flowers, which stop the opening halfway (e.g about 50% opening) and do not complete the opening to 100%.

Furthermore, since Delosperma per stem usually has a lot of flowers, “the flowering state” of Delosperma is not a concept that every flower is blooming. It includes a concept that flowers more than a certain percentage open. This is because the movement of a flower depends on plant conditions, plant size, temperature, intensity of light, and characteristics of a variety. Some flowers complete to open in about 30 minutes, while other flowers complete to open for more than 6 hours. In addition, normally speaking, flower buds emerge one after another with time. Therefore, there are differences between new flowers and old flowers in terms with influences by temperature and light.

Herein, the Delosperma plant according to Embodiment 1 of the present invention is deposited in the following international depositary authority under deposit number FERM BP-22335. The Delosperma plant according to Embodiment 1 of the present invention also includes a progeny of the Delosperma plant, a crossbreeding variety (a hybrid plant), and a progeny of the hybrid plant. The information relating to said deposit will be described as follows.

Kind of deposit: International deposit under the Budapest treaty
Name of the authority: International patent organism depositary, National institute of technology and evaluation

### Address: #120, 2-5-8 Kazusakamatari, Kisarazu-shi, Chiba 292-0818, Japan

Accession number given by the international depositary authority: FERM BP-22335
Identification reference given by the depositor: NISHIKAWA1
Date of the original deposit: Jun. 1, 2017

The embodiment 1 of the present invention provides a new Delosperma plant, which is (a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar, (b) a cultivar produced by crossing species, wherein at least either one of the parents of the species is selected from the Variety A, or (c) a progeny of one of the cultivars (a) and (b). Note that these Delosperma species are basically able to cross with each other and able to generate progenies. This crossing includes self-pollination inside an individual plant and cross-pollination between different individual plants. In case of self-pollination, it uses the pistil of an individual plant and pollens collected from the same individual plant. In case of cross pollination, one parent must be the Delosperma plant related to embodiment 1 of the present invention, while the other parent is not necessary to be such the Delosperma plant as long as it is possible for the other parent to cross with the Delosperma plant according to Embodiment 1 and to generate progenies.

Further, a Delosperma plant relating to embodiment 1 herein is (a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant, and then selecting said cultivar, (b) a cultivar produced by crossing species, wherein at least either one of the parents of the species is selected from the Variety B, or (c) a progeny plant of one of the cultivars (a) and (b).

A method for producing/breeding the Delosperma plant according to embodiment 1 of the present invention has no particular restriction, except that at least either one of parents should be the Delosperma plant characterized by being capable of maintaining a flowering state in case where a room temperature is more than a prescribed temperature, even when it gets dark at nightime. Furthermore, this invention includes a Delosperma plant (product by process) obtained by a method for producing/breeding the Delosperma plant, which has the abovementioned characteristics. The method includes every selecting step/crossing step in order to obtain the Delosperma plant.

Delosperma can be propagated by using seeds, planting cuttings and layering, both in nature and in deliberate cultivation. The Delosperma plant according to embodiment 1 of the present invention can be obtained by utilizing either one of reproduced pollen, ovum, cell, and the genetic information contained in the DNA and RNA of the Delosperma species with abovementioned characteristics. In other words, any reproducible parts of the Delosperma plant can be used for reproducing, and the reproduction is not limited to sexual reproduction but also includes asexual reproduction. Herein, the term “plant(s)” includes plant organs, plant tissues, cells, vegetative propagules and the like, and the plant organs include petals, corolla, flowers, leaves, seeds, fruits, stems, roots, and the like.

The Delosperma plant according to embodiment 1 of the present invention has the characteristics as follows. Firstly, the Delosperma plant is characterized by being capable of maintaining its blooming state in case where an air temperature is more than a prescribed temperature, even when it gets dark at nightime, wherein the prescribed temperature is 15 degree Celsius. As a result of this, the Delosperma plant has a characteristic with much longer opening state per day, as compared with conventional cultivars of Delosperma. Further, the prescribed temperature is preferably 12.5 degree Celsius; more preferably 10 degree Celsius; even more preferable 5 degree Celsius. Note that aforementioned characteristics or a combination of the characteristics of this new Delosperma plant is clearly distinguished from the characteristics of conventional Delosperma species. On the other hand, although it depends on the region, conventional Delosperma species can keep their flowers open from 9 AM in the morning to 3 PM in the afternoon under conditions where they can get enough sunlight and temperature, after the evening, they keep the flowers closing, even in summer. Therefore, it is obvious that the Delosperma plant 1 having abovementioned characteristics is a totally new cultivar.

Herein, the reason why the prescribed temperature ranges from 5 to 15 degree Celsius, which is significant so as to maintain its flowering state, will be described. For example, in Tokyo, the summer gardening season, which has various crops blooming in summer such as petunia, verbena, and so on, is usually from April to October. Although Delosperma has a strong tolerance for cold weather and its roots survive during winter, its flowering season is mainly from spring to autumn, therefore, Delosperma is usually entertained in the summer gardening season. And, for example, Tokyo's minimum temperature in 2015 is 14.5° C. in April, 18.4° C. in October (more specifically, 5.8° C. in January, 5.7° C. in February, 10.3° C. in March, 14.5° C. in April, 21.1° C. in May, 22.1° C. in June, 26.2° C. in July, 26.7° C. in August, 22.6° C. in September, 18.4° C. in October, 13.9° C. in November, 9.3° C. in December). These data show the minimum temperatures in Tokyo. And, Los Angeles in America has almost the same minimum temperatures in terms with gardening season, for example, comparatively colder places such as Amsterdam in Holland, Seattle in America have the minimum temperatures around 10° C.

In other words, it is very rare that the minimum temperature in a warm region becomes under 15° C. during the summer gardening season. Therefore, the Delosperma plant 1, which is capable of maintaining its flowering state even from 5° C. to 15° C., can maintain its flowering state for most of the times in summer, such as in Tokyo and Los Angeles. Further, it is very significant that the Delosperma plant 1 can provide high better outlook for viewers, which can maintain its flowering state even during night or a rainy day.

### EXAMPLES

Next, referring to examples below, the Delosperma plant according to Embodiment 1 will be described. It is to be understood that the examples described herein are not intended to limiting the entire scope of the present invention.

### Example 1

The new Delosperma plant relating to the present invention was invented at a farm located in Okayama Prefecture, Japan, under a breeding program instructed and managed by the inventor. The purpose of this breeding program was to breed a new Delosperma plant having a unique flowering physiology, more specifically, having longer flowering hours than conventional Delosperma species. It is noted that the breeding condition of the new Delosperma plant according to the present invention is not particularly limited to the condition described in Example 1, as long as the condition is applicable to the Delosperma plant.

Firstly, the inventor collected 2,000 of hybrid seeds obtained through crossing of Delosperma cultivars selected from a group consisting of ‘Jewel of Desert Rosequartz,’ ‘Jewel of Desert Ruby,’ ‘Jewel of Desert Garnet,’ ‘Jewel of Desert Moon Stone,’ ‘Jewel of Desert Topaz,’ ‘Jewel of Desert Peridot,’ ‘WOW (Wheels of Wonder) DRW5,’ ‘WOWDRW7,’ ‘WOWDOY3,’ ‘WOWDAY2,’ ‘WOW2011-1,’ ‘WOWDRY1,’ ‘DSAM13-1,’ ‘DSAA13-1’ and ‘DSAB13-1’ owned by the inventor, and bred them in a greenhouse near the inventor's house located at Katsuta-gun in Okayama Prefecture, Japan, with ordinary breeding methods. After having crossed the abovementioned seeds with various seeds owned by the inventor for three generations repeatedly, a single mutant individual of a Delosperma plant with a unique characteristic, which can maintain its flowering state even when it gets dark and it becomes low temperature, was suddenly obtained.

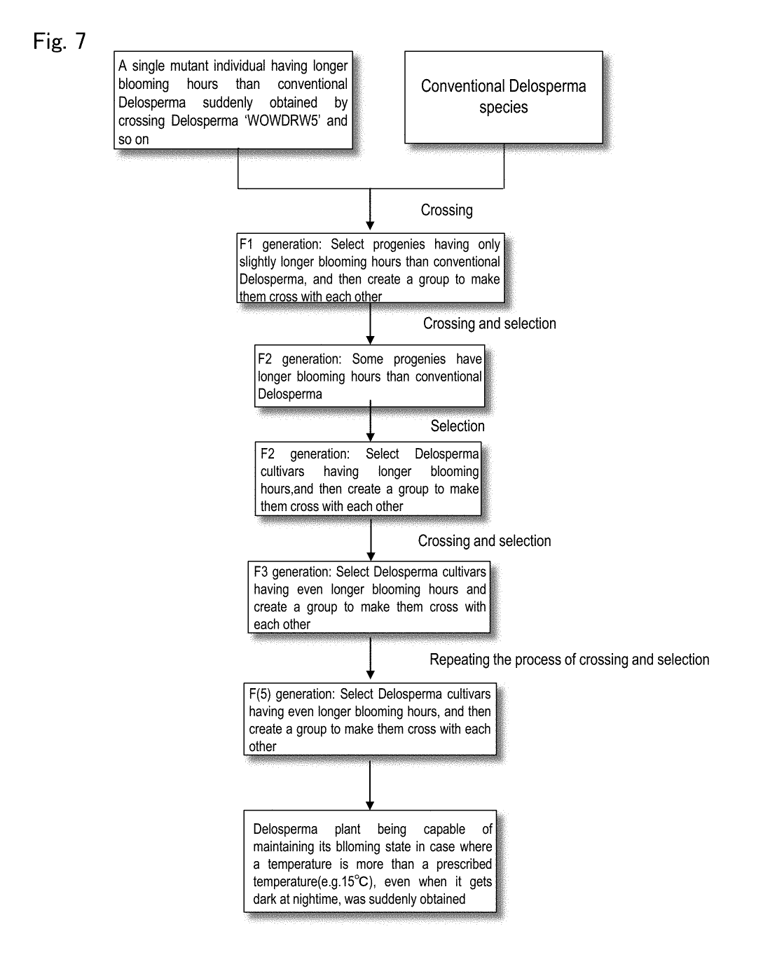

Next, after having repeated crossing and selection processes using the cultivars from a group of progenies obtained from the single mutant individual, a Delosperma plant being capable of maintaining a flowering state even when it gets dark and low temperature was successfully obtained. To be more specifically, as described in FIG. 7, the procedure is as follows: (1) crossing the mutant individual of Delosperma, which is capable of maintaining a flowering state even when it gets dark and it becomes low temperature. The mutant Delosperma plant was suddenly obtained by crossing Delosperma species selected from a group of Delosperma ‘WOWDRW5’ and so on; (2) next, collecting seeds from the Delosperma plant, and selecting Delosperma cultivars having longer blooming hours obtained by breeding the seeds. And crossing said Delosperma cultivars, and then selecting Delosperma cultivars having much longer blooming hours (repeating crossing and selection) for multiple generations in order to gradually enhance the characteristic of having longer blooming hours. However, it is noted that when Delosperma cultivars are grown from seeds, the flowering hours of Delosperma is not stable. For example, the ratio of cultivars with longer blooming hours was sometimes below 10% and sometimes over 30% in the next generation. When reaching a stage that some of Delosperma cultivars have longer blooming hours, a Delosperma plant being capable of maintaining a flowering state in case where a room temperature is more than a prescribed temperature, even when it gets dark at nightime, wherein the prescribed temperature is 15 degree Celsius, preferably 12.5 degree Celsius, more preferably 10 degree Celsius, even more preferably 5 degree Celsius, was suddenly emerged. And (3) genetically fixing a desired characteristic (being capable of maintaining its flowering state in case where a room temperature is more than a prescribed temperature, even when it gets dark at nightime) by repeating crossing and selection of the Delosperma plant having said characteristic even further for multiple generations. In other words, the Delosperma plant with the desired characteristic can surely be obtained, even in small amount, by utilizing the crossed Delosperma plant (as at least either one of the parents for crossing) within multiple generations (which means that the characteristic is repeatable).

As described above, the characteristic of the Delosperma plant according to embodiment 1 of the present invention is “repeatable (inheritable)”, which means that the same result can be repeatable if the same breeding method is repeated, and the Delosperma plant is clearly different from a conventional plant in its characteristic (flowering physiology). In addition, the invertor has already obtained more than 100 varieties of Delosperma having this characteristic by utilizing the Delosperma plant, which was initially obtained and was capable of maintaining its flowering state in case where a room temperature is more than a prescribed temperature, even when it gets dark at nightime. This also indicates that the characteristic according to the present invention is repeatable.

### Example 2

Secondly, a crossing experiment was implemented. Specifically, the Delosperma plant with the specific characteristic herein had been bred and the pollens from stamens of Delosperma with having normal blooming hours were deposited into its pistil of the Delosperma plant. The Delosperma plant was bred until it generated seeds, and then the seeds collected from the plant were sowed and bred. As a result, the Delosperma plants being capable of maintaining its flowering state in case where an air temperature is more than a prescribed temperature, even when it gets dark at nightime, wherein the prescribed temperature is 15 degree Celsius, preferably 12.5 degree Celsius, more preferably 10 degree Celsius, even more preferably 5 degree Celsius, were found in the first generation (a child generation), the next generation (a grandchild generation), and the next next generation obtained through the said breeding procedures. Herein, the percentage of these Delosperma plants was sometimes below 10% and sometimes over 30%.

Accordingly, by means of depositing pollens from fertile commercial cultivar into its pistil of the Delosperma plant having said flowering physiology according to embodiment 1 of the present invention, the Delosperma having the flowering physiology will be produced in following generations, and hence this shows that this characteristic is inheritable. As described above, in Example 2, the Delosperma plant with the desired characteristics can surely be obtained by utilizing the Delosperma plant (as at least either one of the parents for crossing) within multiple generations.

### Example 3

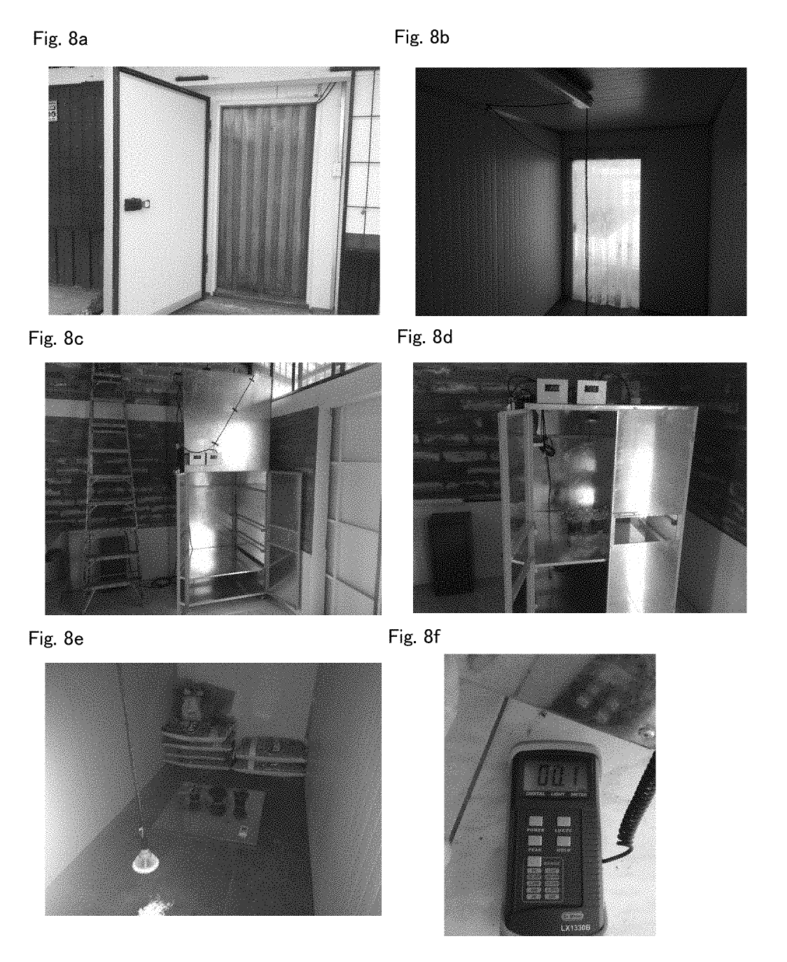

Next, referring to FIG. 8 to FIG. 10, the details of the Delosperma plant being capable of maintaining its flowering state in case where an air temperature is more than a prescribed temperature, even when it gets dark at nightime will be explained as follows. Herein, the experiments, which relate to the flowering physiology of Delosperma according to given examples, were conducted in a laboratory equipped for biological environment control, which is called biotron shown in FIG. 8. In this biotron (Height 2 m, width 2.5 m, depth 5 m, and Height 2.5 m, width 1.2 m, depth 1.2 m), basic conditions such as temperature and light, which are necessary for survival and breeding of plants. These are artificially controlled so that biological research can be achieved. Furthermore, for example, the illumination level when achieving dark at nightime in the biotron was 0.1 luce.

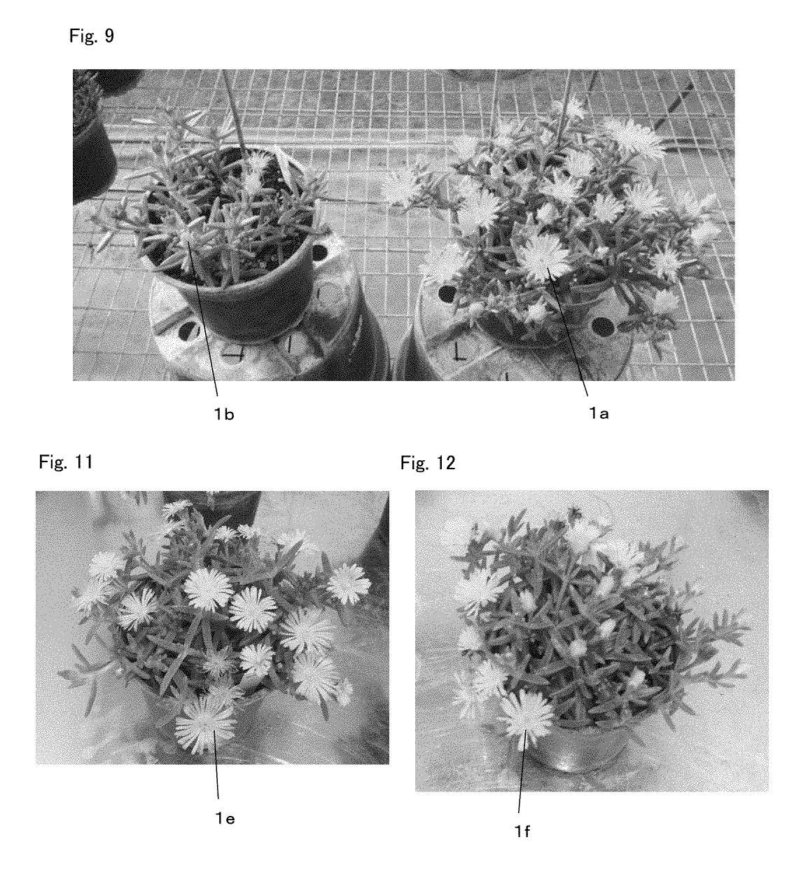

As shown in FIG. 9, it is noted that the plant on the right has grown well while the plant on the left has not grown yet, even if they are of the same variety. In this case, it's likely to occur that the flower of 1a blooms while the flower of 1b closes even under the same temperature. For example, flower 1a of Delosperma blooms at 3 degree Celsius lower temperature, as compared with flower 1b of Delosperma. Accordingly, the flowering physiology has to be examined by making use of plants which have grown enough.

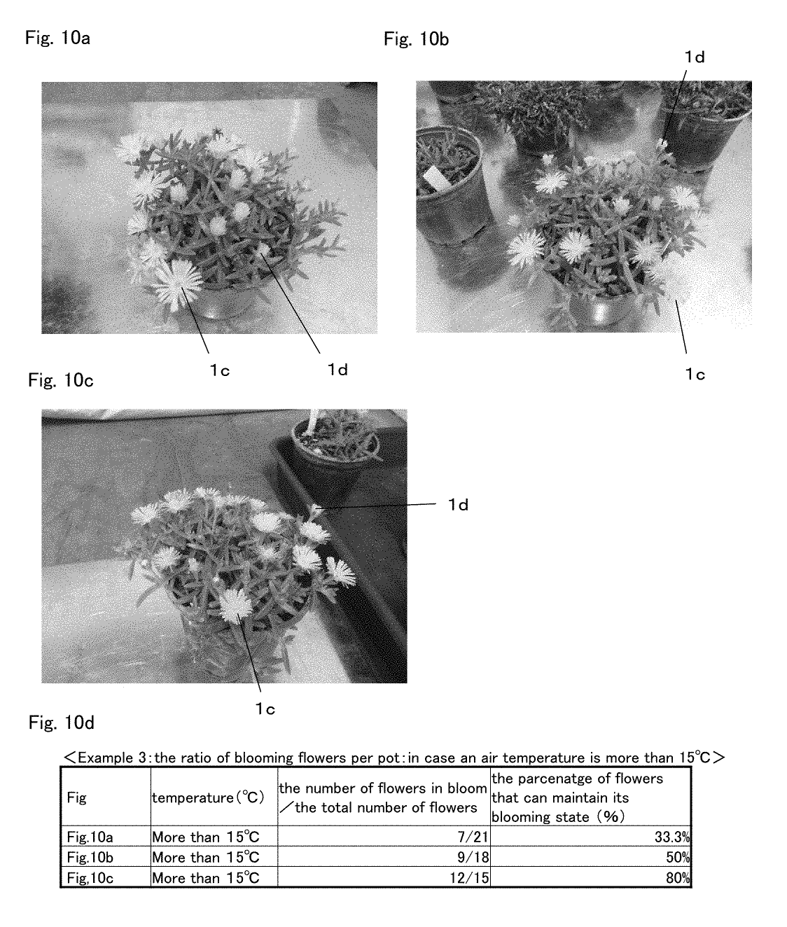

Example 3, shown in FIG. 10, pertains to the Delosperma plant being capable of maintaining its flowering state in case where an air temperature is more than a prescribed temperature, even when it gets low illuminance. Specifically, the Delosperma plant is capable of maintaining a flowering state in case where the temperature is more than 15 degree Celsius, even when the illuminance is gradually reduced less than 1 luce.

FIG. 10a to FIG. 10c show that some flowers 1c open, while other flowers 1d close. Specifically, shown in FIG. 10d, the number of flowers in blooming state per pot was 7, while the total number of flowers was 21 in FIG. 10a. Therefore, the percentage of flowers that can maintain its blooming state during nightime was 33%. In FIG. 10b, the number of flowers in blooming state per pot was 9, while the total number of flowers was 18. Therefore, the percentage of flowers that can maintain its flowering state during nightime was 50%. In FIG. 10c, the number of flowers in blooming state per pot was 12, while the total number of flowers was 15. Therefore, the percentage of flowers that can maintain its flowering state during nightime was 80%.

This is because there is a natural tendency for flowers, which have just started blooming, to have difficulty maintaining its flowering state at low temperature. On the other hand, there is a natural tendency for flowers, which are approaching its longevity, to easily maintain its flowering state even at low temperature. For example, if the longevity of flowers is around 14 days, the flowers will tend to close during night time for the first 7 days, and the flowers will tend to open during night time for the last 7 days. In other words, old flowers, which are close to its longevity, can easily maintain its flowering state. As a result of this, the ratio of flowers that can maintain its flowering state during night time is changing, depending on the ratio of old flowers per stem. Therefore, the Delosperma plant has both flowers that can maintain flowering state and cannot maintain flowering state at the same temperature even in the same stem. Further, although the ratio of old flowers to new flowers depends on the environment and its management method, in the event that the Delosperma plant is managed under preferable conditions, the ratio of old flowers is more than 30% in general. Thus, in this invention, the ratio of flowers, which can maintain its flowering state during nightime, is at least more than 20% per stem, more preferable 40%. Note that flower longevity is easily influenced by temperature, moisture, getting wet by rain, and so on, which may result in shorter or longer longevity.

### Example 4

Next, referring to FIG. 11 to FIG. 14, the Delosperma plant being capable of maintaining a flowering state according to Example 4 in case where an air temperature is more than a prescribed temperature, even when it gets artificially dark in the biotron will be explained.

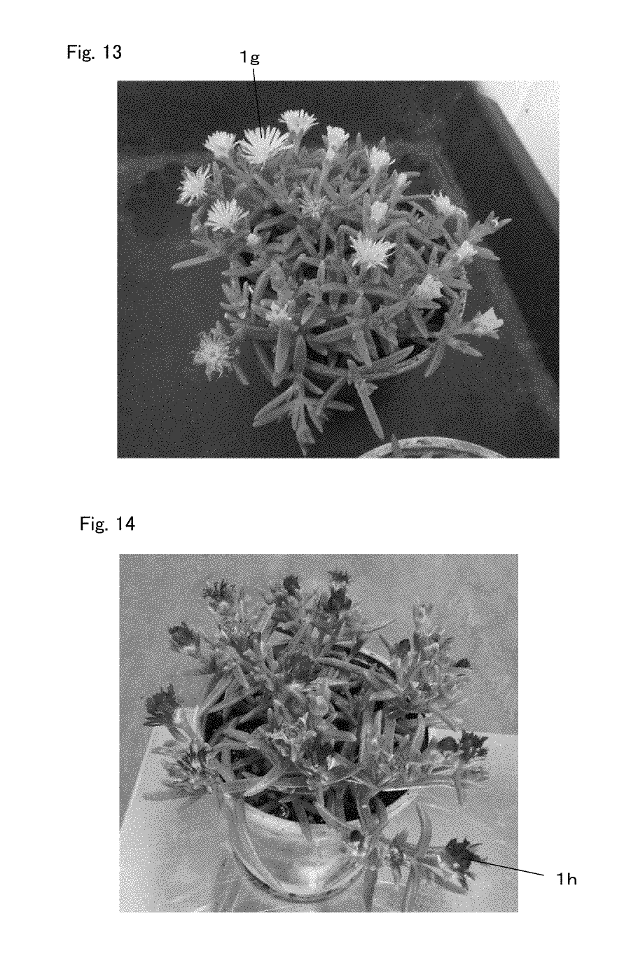

The Delosperma plants shown from FIG. 11 to FIG. 13 are the same kind. FIG. 11 shows the Delosperma plant 1e being capable of maintaining its flowering state under 15 degree Celsius. FIG. 12 shows the Delosperma plant 1f being capable of maintaining its flowering state under 10 degree Celsius. And FIG. 13 shows the Delosperma plant 1g being capable of maintaining its flowering state under 5 degree Celsius.

FIG. 11 shows the Delosperma flowers 1e under 15 degree Celsius. Herein, except for flowers having expired its longevity and flower buts, when conditions are right, the flowers that can maintain its flowering state during nightime under 15 degree Celsius were observed in this FIG. 11. In this state, the temperature was decreased to 10 degree Celsius. FIG. 12 shows the Delosperma flowers 1f that can maintain its flowering state during nightime under 10 degree Celsius. However, the ratio of blooming flowers was decreased, as compared with FIG. 11. In this state, the temperature was further decreased to 5 degree Celsius. FIG. 13 shows the Delosperma flowers 1g during nightime under 5 degree Celsius. In this case, there was no flower that could maintain its flowering state, and there were only flowers whose opening were 30 to 50%. Herein, as described above, flower in bloom means flowers having over 50% opening.

Accordingly, the lower temperature becomes, the less flowers per pot can maintain its flowering state. In addition to this, when temperature reaches very low, most flowers close. Generally speaking, new flowers start to close at first as temperature becomes lower (except for flowers having expired its longevity and flower buts). In other words, it is thought that new flowers start to close at first, and then old flowers start to close.

Furthermore, as shown in FIG. 14, all flowers close when the temperature decreases more (lower than 5 degree Celsius). However, sometimes the old flower 1h, which have opened once, keeps opening under the circumstances.

### Example 5

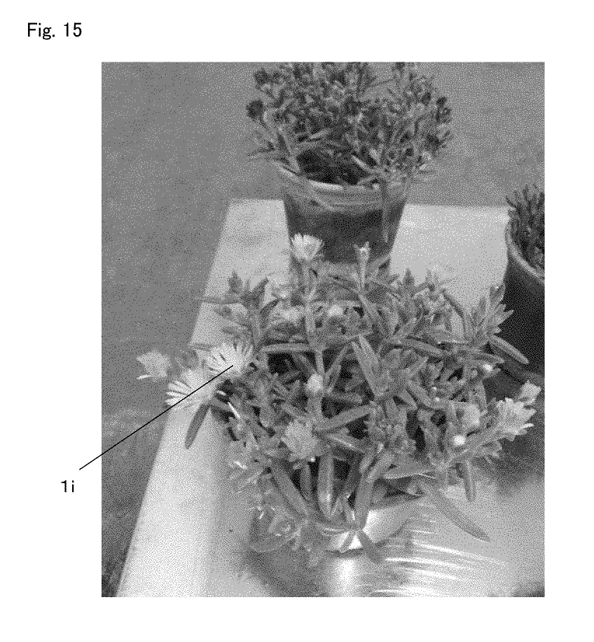

Next, referring to FIG. 15, a Delosperma plant being capable of maintaining a flowering in case where the temperature is less than 5 degree Celsius, even when it gets dark, will be explained. Sometimes, some flowers such as a flower 1i in FIG. 15 can maintain its flowering under less than 5 degree Celsius. However, the ratio of opening flowers is very low, and sometimes all flowers close under the circumstances.

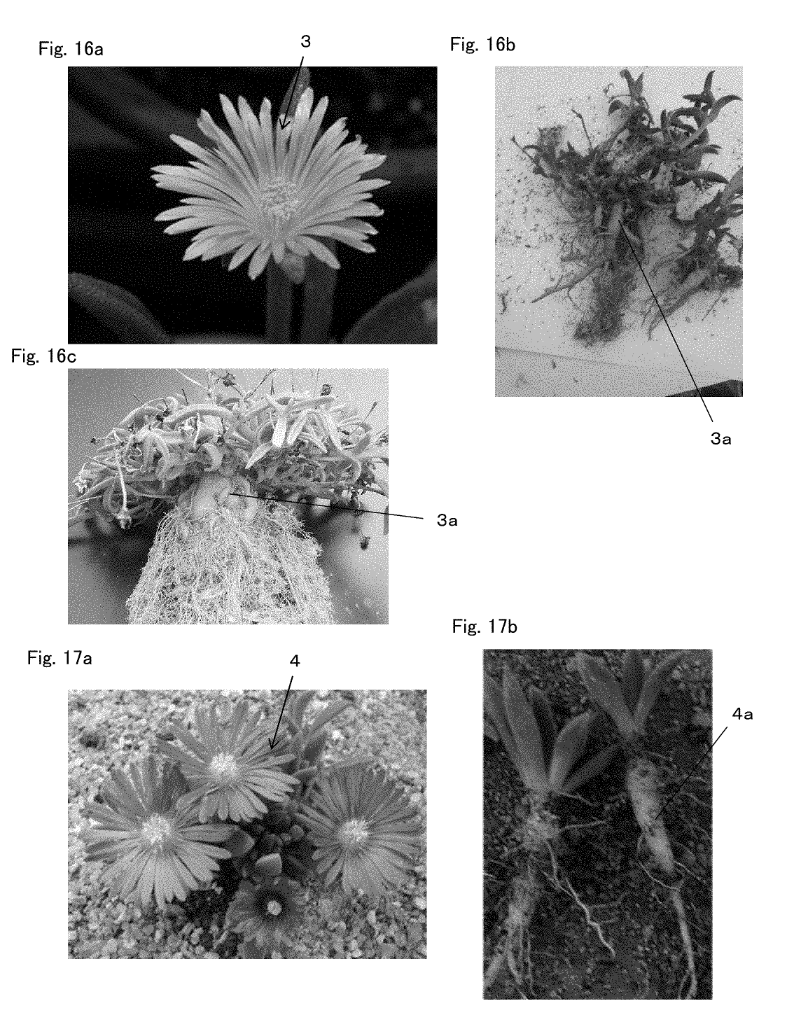

Next, the details of roots of the Delosperma plant according to embodiment 1 of the present invention will be explained, referring to FIG. 16 and FIG. 17. Because, the root parts of the Delosperma plant are totally different from those of Delosperma sutherlandii and Delosperma ashtonii.

The Delosperma plant according to embodiment 1 of the present invention is also characterized by its roots which becomes capillary roots and does not form tuberous roots. On the other hand, as shown in FIG. 16b, FIG. 16c, and FIG. 17b, Delosperma sutherlandii 3 and Delosperma ashtonii 4 have tuberous roots 3a,4a just like carrot so as to retain nutrition and water in it. However, the structure of root parts of such cultivars is obviously different from the Delosperma plant according to embodiment 1 of the present invention.

Furthermore, hybrid species were not able to be collected during the crossing experiment between the Delosperma plant relating to embodiment 1 of the present invention and Delosperma sutherlandii 3. Although the Delosperma plant according to embodiment 1 of the present invention botanically belongs to the same variety with Delosperma sutherlandii 3 and Delosperma ashtonii 4, it cannot be used for crossing with them, and the characteristic of root parts is obviously different as described above. It means that, genetic information of Delosperma cultivars which form tuberous roots such as Delosperma sutherlandii 3 and Delosperma ashtonii 4 are not used for producing the Delosperma plant herein.

As described above, the Delosperma plant according to embodiment 1 of the present invention is characterized by being capable of maintaining its flowering state in case where a room temperature is more than a prescribed temperature, even when it gets dark at nightime, wherein the prescribed temperature is 15 degree Celsius. Further, the prescribed temperature is preferably 12.5 degree Celsius; more preferably 10 degree Celsius; even more preferable 5 degree Celsius. As a result, the present invention can provides the new Delosperma plant that can keep the flowers opening, regardless of the temperature and weather such as a solar radiation value. And the Delosperma plant can keep opening during open hours in flower shops. Therefore, the Delosperma plant can enhance its commercial value, as compared with conventional Delosperma species. It is to be understood that the abovementioned Delosperma plant described herein is not intended to limiting the scope of morphological and ecological characteristics of Delosperma, except for the flowering physiology and the characteristic of root parts.

### Embodiment 2

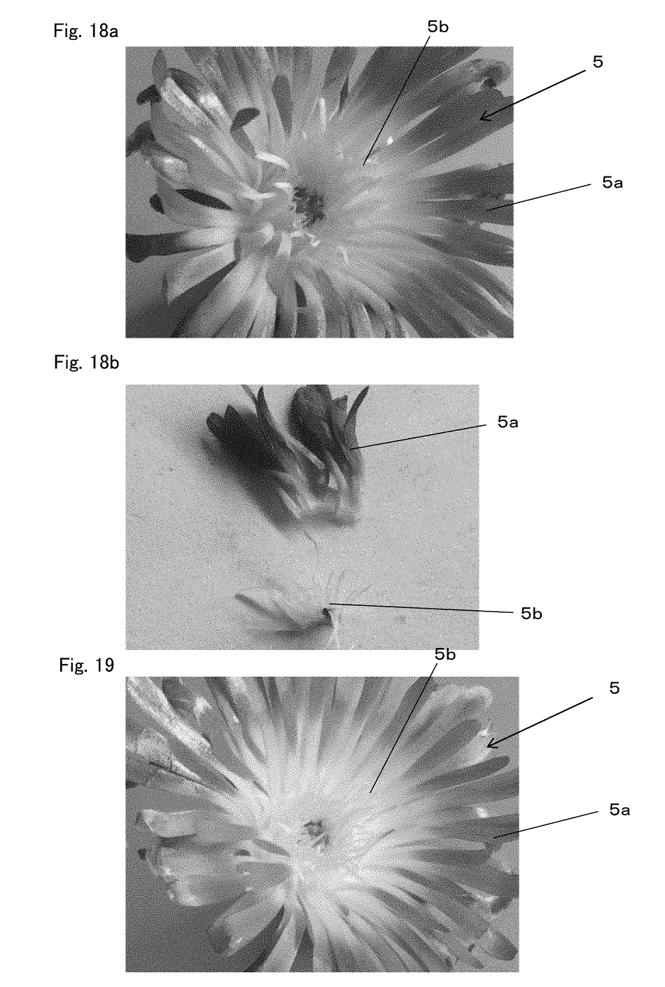

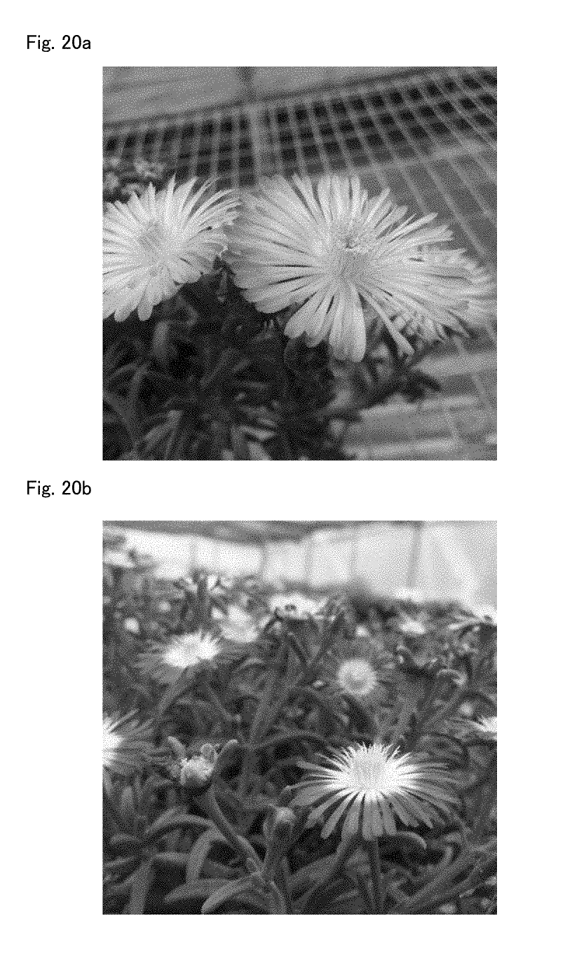

Referring from FIG. 18 and FIG. 19, a Delosperma plant according to Embodiment 2 of the present invention will be described.

The following characteristics have been represented and observed at the Delosperma plant according to embodiment 2 of the present invention. The Delosperma plant described herein has the characteristics of not having stamens with anther in addition to having longer blooming hours per day. On the other hand, conventional Delosperma species usually have several tens of stamens. Therefore, it can be said that the Delosperma plant according to embodiment 2 of the present invention with characteristics of having longer blooming hours per day and not having stamens with anther is a new cultivar with completely new characteristics. These characteristics and the combination of these characteristics are clearly distinguished as a new Delosperma variety from the conventional Delosperma varieties.

The double-flower type Delosperma species being capable of maintaining its flowering state when it get dark and not having stamens with anther (hereinafter referred to as “Delosperma 5”) are shown in FIG. 18 and FIG. 19. It can be recognized that all stamens of Delosperma 5 are converted and grown into petals 5a and petaloids 5b. Because the Delosperma plant described herein has longer blooming hours per day than conventional Delosperma species, it is more commercially valuable. In addition to this, since the Delosperma plant is a male sterile plant, which can avoids seeds from being produced, and therefore the Delosperma plant has larger number of blooms. As a result of this, it is clear that the Delosperma plant according to embodiment 2 has more commercial values.

It is to be understood that the abovementioned Delosperma plant described herein is not intended to limiting the scope of morphological and ecological characteristics of Delosperma, except for blooming hours, having no stamen, and the characteristic of root parts.

### EXAMPLE

Next, an example of the Delosperma plant according to the second embodiment 2 of the present invention will be explained as follows. However, it is noted that the form of present invention is not limited to the described practical example.

Similar to practical examples described in embodiment 1 of the present invention, the selection procedure is as follows: (1) crossing above mentioned mutant individual of Delosperma, which has longer blooming hours than its parents. (2) next, collecting seeds, and selecting Delosperma cultivars having even longer blooming hours obtained by breeding the seeds. And crossing said Delosperma cultivars, and then selecting Delosperma cultivars having longer blooming hours (repeating crossing and selection) for multiple generations in order to gradually enhance the characteristic of having longer blooming hours. In this example, the Delosperma cultivars with characteristics of not having (or having a fewer) stamens with anther in addition to having longer blooming hours were selected.

It has been observed that, because this Delosperma plant is a male sterile plant that has no stamen and no anther, the seeds are not likely to be produced unless otherwise a non-self pollen has been deposited from another Delosperma plant.

It is to be noted that the Delosperma plant can be proliferated (asexually reproduced) with herbaceous cutting and other techniques. Herbaceous cutting can be conducted with known methods for a person having ordinary skill in the art. For example, after a tissue cut from Delosperma have been cultivated under the optimal environments in order to produce a plantlet having roots and shoots, and then the plantlet is raised under the environments. The tissue can also be cultivated through mericloning which extracts approximately 1 millimeter of growing point from sprout and cultivates it in sterile culture.

As described above, the Delosperma plant according to embodiment 2 of the present invention can maintain its flowering state even when it gets dark and does not have stamens. Therefore, the Delosperma plant has longer flowering hours and the number of blooms per stem increases, because the Delosperma plant is a male sterile plant, and its appearance becomes more contact. Hence, these characteristics can enhance the commercial value of the Delosperma plant.

### Embodiment 3

A Delosperma plant according to Embodiment 3 of the present invention will be described. The Delosperma plant according to embodiment 3 of the present invention has an infertile pistil and unable to generate seeds even when the pistil receives fertile pollens. The self-incompatibility, the reaction that a pistil rejects self pollen to avoid inbreeding, is generally known. However, the Delosperma plant described herein is unable to generate seeds through receiving self pollens (self-pollination) and non-self pollens (external pollination). In other words, the Delosperma plant has the characteristic of a female sterile flower, which is unable to generate seeds even when its pistil receives self or non-self pollens. As a result of this, the Delosperma plant comprises a female sterile flower has more numbers of bloom per stem, and this characteristic makes the Delosperma plant more commercially valuable. Herein, the Delosperma plant mentioned in embodiment 2 of the present invention has no pollen in its stamens, but its pistil can have fertility, and can generate seeds if pollens are deposited from an external plant by chance. On the other hand, the Delosperma plant according to embodiment 3 of the present invention has a female sterile flower having an infertile pistil. Therefore, seeds will never be generated even when it receives any type of pollens.

Such a female sterility is also resulted from mutation and such a characteristic has been enhanced and fixed by repeating crossing and selection for multiple generations. Such a characteristic is repeatable at the grandchild generation (e.g. for around 10%) at the experimental station of the inventor.

The present invention is not limited to the configuration of the above embodiments and various modifications can be made within a scope not changing the gist of the present invention. For example, it is possible to breed Delosperma having said flowering physiology and colorful flowers by crossing the Delosperma plants according to the above embodiments with Delosperma cultivars having a flower with different colors.

Furthermore, it can be expected that genes in the DNA and RNA relating to the characteristics of the flowering physiology, having no stamens or a female sterility will be extracted from abovementioned Delosperma plants and embedded the genes into other cells. This will lead to reproducing other plants acquiring such DNA and RNA, and to creating plants having these characteristics other than Delosperma.

1. A Delosperma plant characterized by being capable of maintaining a flowering state in case where an air temperature is more than a prescribed temperature, even when it gets dark at nightime,
wherein the prescribed temperature is 15 degree Celsius.

2. The Delosperma plant of claim 1, wherein the prescribed temperature is 12.5 degree Celsius.

3. The Delosperma plant of claim 1, wherein the prescribed temperature is 10 degree Celsius.

4. The Delosperma plant of claim 1, wherein the prescribed temperature is 5 degree Celsius.

5. The Delosperma plant of claim 1, wherein said dark at nightime indicates a space where illuminance is reduced from daylight to less than 1 luce.

6. The Delosperma plant of claim 1, wherein a ratio of the flowers, which are capable of maintaining the flowering state, is at least more than 20% of all the flowers per stem.

7. The Delosperma plant of claim 1, wherein a ratio of the flowers, which are capable of maintaining the flowering state, is at least more than 40% of all the flowers per stem.

8. The Delosperma plant of claim 1, wherein the Delosperma plant comprises no stamen.

9. The Delosperma plant of claim 1, wherein the Delosperma plant comprises a female sterile flower, which is unable to generate seeds even after a pistil of the Delosperma plant receives self or non-self pollens.

10. The Delosperma plant of claim 1, wherein the Delosperma plant forms no tuberous root.

11. The Delosperma plant of claim 1, wherein the Delosperma plant is
(a) a cultivar called Variety A, which is produced by obtaining an individual mutant from hybridizing at least one of the individuals selected from a group consisting of Delosperma cooperi, Delosperma nubigenum, Delosperma coungestum, Delosperma esterhuysenisa, Delosperma floribundum, Delosperma aberdeenense and Delosperma ‘John Proffitt’, and by crossing using the individual mutant and then selecting said cultivar,
(b) a cultivar produced by crossing species, wherein at least either one of the parents of the species is selected from the Variety A, or
(c) a progeny of one of the cultivars (a) and (b).

12. The Delosperma plant of claim 1, wherein the Delosperma plant is
(a) a cultivar called Variety B, which is produced by obtaining an individual mutant from hybridizing at least one of the individuals selected from a group consisting of ‘Jewel of Desert Rosequartz’ (U.S. Plant Pat. No. 23,452), ‘Jewel of Desert Ruby’ (U.S. Plant Pat. No. 23,453), ‘Jewel of Desert Garnet’ (U.S. Plant Pat. No. 23,471), ‘Jewel of Desert Moon Stone’ (U.S. Plant Pat. No. 23,491), ‘Jewel of Desert Topaz’ (U.S. Plant Pat. No. 23,492), ‘Jewel of Desert Peridot’ (U.S. Plant Pat. No. 23,566), ‘WOWDRW5’ (U.S. Plant Pat. No. 25,572), ‘WOWDRW7’ (U.S. Plant Pat. No. 25,599), ‘WOWDOY3’ (U.S. Plant Pat. No. 25,600), ‘WOWDAY2’ (U.S. Plant Pat. No. 25,645), ‘WOW2011-1’ (U.S. Plant Pat. No. 25,684), ‘WOWDRY1’ (U.S. Plant Pat. No. 26,157), ‘DSAM13-1’ (U.S. Plant Pat. No. 27,013), ‘DSAA13-1’ (U.S. Plant Pat. No. 27,014) and ‘DSAB13-1’ (U.S. Plant Pat. No. 27,056), and by crossing using the individual mutant and then selecting said cultivar,
(b) a cultivar produced by crossing species, wherein at least either one of the parents of the species is selected from the Variety B, or
(c) a progeny plant of one of the cultivars (a) and (b).

13. The Delosperma plant of claim 1, wherein the Delosperma plant is one of varieties selected from a Delosperma plant deposited with the international depositary authority under deposit number FERM BP-22335, a progeny of said Delosperma plant, a hybrid plant of the Delosperma plant, and a progeny of the hybrid plant.

14. A Delosperma plant obtained by utilizing either one of pollen, ovum, cell, and genetic information relating to DNA and RNA of the Delosperma plant of claim 1.

15. A method for producing a Delosperma plant comprising;
(a) utilizing either one of pollen, ovum, cell, and genetic information relating to DNA and RNA of the Delosperma plant of claim 1 so as to produce the Delosperma plant.
